(19) United States
(12) Patent Application Publication
(10) Pub. No.: US 2012/0027747 A1
(43) Pub. Date: Feb. 2, 2012

(54) Compositions and methods for mycotoxin decontamination of animal feed, food, soil and plants using biomass of filamentous fungi and its selected components

(75) Inventors: Dennis Tranquil, Jupiter, FL (US); Albert Vladimirovich Kanarsky, Volzhsk (RU); Elizabeth Tranquil, Jupiter, FL (US); Zosya Albertovna Kanarskaya, Kazan (RU); Oleg Nickolaevich Okunev, Puschino (RU)

(73) Assignee: CUBENA, INC., Jupiter, FL (US)

(21) Appl. No.: 12/899,464

(22) Filed: Oct. 6, 2010

Related U.S. Application Data
(60) Provisional application No. 61/249,597, filed on Oct. 7, 2009.

(51) Int. Cl. A61K 36/06

(52) U.S. Cl. 424/115; 424/195.16

(57) ABSTRACT

A method of rendering harmless mycotoxins contaminating food, animal feed and assisting infection of plant hosts by microbial parasites is proposed. The method comprises binding mycotoxins by a novel adsorbent, consisting partially or in full of the biomass of filamentous fungi or isolated fungal biomass components, such as chitin, chitozan and hyrdophobins, or fungal biomass enriched in its capacity to bind said mycotoxins using fungal species and strain selection, genetic engineering of fungi, modification of fungal fermentation conditions and downstream physical and chemical treatment of fungal biomass, such as milling or micronization in a dry state. The resulting adsorbent can bind a wide range of mycotoxins, including the ones difficult to bind (Ochratoxin, T-2, DON, NIV) using a current generation of mycotoxin adsorbents based on clay, resins, yeast and bacterial biomass. The adsorbent can be used as an animal feed additive, functional food additive and agricultural fungistatic/fungicide.

### REFERENCES CITED

U.S. Patent Documents

| | | |
|---|---|---|
| U.S. Pat. No. 6,045,834 | April 1999 | Howes A. D. and |
| | | Newman K. E. |
| U.S. Pat. No. 7,514,085 | April 2009 | Kristiansen B. |
| US application 20090060965 | March 2009 | Lyn M. et al. |
| US application 20100239537 | September 2010 | Zhou T. et al. |
| US application 20100028303 | February 2010 | Martin W. R., Jr. and |
| | | Hayes C. K. |
| US application 20100189856 | December 2008 | Tranquil E. et al. |
| US application 20100178300 | July 2010 | Yiannikouris A. and |
| | | Thielen U. A. |

Foreign Patent Documents

| | | |
|---|---|---|
| WO/1998/034503 | August 1998 | El-Nezami H. et al. |
| WO/2007/116245 | October 2007 | Giusto G. |
| EP 08152315.1 | March 2008 | Viksoe-Nielsen A., |
| | | Soeerensen B. H. |

### FIELD OF THE INVENTION

The present invention is addressing the problem of mycotoxin decontamination of the animal feed, human food and site of plant host invasion by fungal infection using the effect of binding the mycotoxins by a novel adsorbent. The adsorbent contains an organic component based on biomass of filamentous fungi with optional addition of conventional non-proprietary mycotoxin binding agents known in the art. As a result, the mycotoxins are safely excreted from the digestive tract of the agricultural and companion animals or humans without detrimental effects on animal performance or human wellbeing. The binding composition is also useful while administering into soil and onto seeds, seedlings and plants to reduce and overt the invasion of parasitic fungi upon agricultural plants.

### BACKGROUND OF THE INVENTION

The biotechnology based solutions to bind and render harmless mycotoxins in food, feed and agriculture include the use of yeast cell wall (U.S. Pat. No. 6,045,834 and US patent application 20100178300), specially selected bacteria (WO/1998/034503, US patent application 20100239537) and plant biomass (US patent application 20100189856).

Fungal cultures have been known as producers of mycotoxin-degrading enzymes, both hydrolases and oxidases (EP 08152315.1) and of immuno modulating cell wall fragements (U.S. Pat. No. 7,514,085). Filamentous fungi are also used as probiotics to outcompete parasitic and mycotoxin-producing fungi (WO/2007/116245, US patent applications 20090060965 and 20100028303).

However, the affinity of fungal biomass and its fragments towards mycotoxins and the potential use of the former as mycotoxin binding components in food, animal feed and agriculture have not been reported.

### SUMMARY OF THE INVENTION

A primary object of the present invention is to provide a method for adsorbing and consequent rendering harmless of mycotoxins present in common animal feedstuffs, human foods and the ones assisting in infection of agricultural crops by fungal parasites. The method comprises use of a novel mycotoxin binder based on the biomass of filamentous fungi. The biomass is either derived as an abundant by-product from a number of industrial fermentation processes (such microbial production of citric acid, antibiotics, enzymes, vitamins and pigments), or from industrial processes where fungal biomass is a part of a bioconversion mixture (such as enzymatic conversion of biomass into biofuels with direct use of fungal fermentation broth for bioconversion). Alternatively, the biomass of filamentous fungi, functionally enriched fungal biomass or its components (i.e. the components responsible for binding of mycotoxins) could be produced by targeted fermentation.

In both cases the affinity and capacity of the mycotoxin binder based on the biomass of filamentous fungi can be further improved using fungal species screening with selection of the optimal producer, strain selection, genetic engineering of fungi, modification of fungal fermentation conditions and downstream physical and chemical treatment of fungal biomass.

The object of the present invention is to provide a composition comprising a combination as described above which may render harmless a wider range of multiple mycotoxins, with specific emphasis on mycotoxins typical for Northern climates (OTA, T-2, DON, NIV), currently poorly handled by the existing mycotoxin adsorbents, in addition to mycotoxins typical for Southern climates (AF, FUM, ZEN), that are handled efficiently by the current generation of mycotoxin binders.

The invention proposes a method of mycotoxin sequestration, where the compositions provided by the invention are fed to any agricultural, aquacultural, companion and wild animal. When admixed with feed or fed as a supplement, the compositions with their surprisingly increased mycotoxin-binding capacity and expanded mycotoxin type range, decrease intestinal absorption of the mycotoxins by the affected animal, thereby improving performance and health, and reducing the incidence of mycotoxin-associated diseases.

The invention also proposes a method of mycotoxin sequestration, where compositions provided by the invention can be added to any human food, dry, powder, formed, paste, jelly or liquid or used as a functional nutritional supplement in the form of powder, tablets, paste or suspension, with or without other ingredients. When admixed with food or fed as a supplement, the compositions with their surprisingly increased mycotoxin-binding capacity and expanded mycotoxin type range, decrease intestinal absorption of the mycotoxins by the affected humans, thereby improving performance and health.

The invention also proposes a method of mycotoxin sequestration in agriculture, where the composition can be added to agricultural soil, applied on seeds, seedlings, plants and crops (including fruit and vegetables) to provide a resistance to fungal infections to the plants and crops.

### DETAILED DESCRIPTION OF THE INVENTION

The present invention is based upon the surprising discovery that the biomass material derived from filamentous fungi can have an unexpected binding effect on mycotoxins, including mycotoxins of Northern origin, which are known to be difficult to sequester until now. Thus, the invention provides a method and a composition for binding mycotoxins present in animal feeds, food and during plant host fungal infection interactions. The method utilizes a combination of unmodified or modified biomass of filamentous fungi and an optional non-proprietary mycotoxin binding agent or multiple agents known in the art.

The biomass can be derived either from a mainstream industrial fungal fermentation, typically performed by a third party for its own purposes, such as, but not limited to: production of enzymes, other peptides, secondary metabolites, pharmaceuticals, nutraceuticals, acids, fatty acids, fats and their derivatives, polysaccharides, flavors, vitamins, pigments. Alternatively, the biomass of interest can be derived from a dedicated in-house fermentation, specifically targeting the biosynthesis of mycotoxin-binding agents as primary products.

The preferred mainstream fungal fermentation processes yielding the flamentous fungi biomass by-product include, but are not limited to: citric acid fermentation, glucoamylase fermentation, fungal pectinase, fungal protease, fungal peptidase and fungal amylase fermentation, fermentation of Trichoderma, Aspergillus, Rhizopus, Penicillium, Humicola and Chrysosporium/Myceliophthora for cellulase, beta-glucanase and xylanase, production of antibiotics, vitamins and pigments (carothene, astoxanthine), omega-unsaturated fatty acids, also fermentations where genetically modified fungal hosts are used to produce proteins, peptides and secondary metabolites.

The preferred targeted fermentation species belong to, but not limited to, genera of Aspergillus, Rhizopus, Trichoderma, Penicillium, Acremonium, Monilia, Chryzosporium/Myceliophthora, Chaetomium, Geotrichum, Mucor, Neurospora, and Fusarium mycromycetes and to basidiomycota (basidiomycetes), preferably the ones successfully converted to submerged fermentation.

When running dedicated fermentations to produce the biomass of filamentous fungi (opposite to using by-product biomass available elsewhere as-is), the mycotoxin binding properties of the biomass can be further enhanced. In the preferred embodiment of the invention, measures are taken to maximize the production of chitin and hydrophobins.

The production of Chitin and Chitosan in enhanced creating the best conditions of biomass growth with ample supply of Oxygen, Nitrogen and Carbon nutrients and with high shear mixing in submerged fermentation. In addition, a fungal species or strain can be selected with elevated production of an enzyme system converting Chitin into Chitosan. In solid state fermentation measures taken to enhance Chitin and Chitosan production include supplementation of solid substrate with Biotin, Niacin, starch, other sources of readily available sugars, urea and ammonium sulfate as source of Nitrogen.

The production of Hydrophobins can be enhanced subjecting the microorganism to stress, such as transfer from submerged fermentation to solid state fermentation for 6-12 hours, deprivation of Carbon or Nitrogen nutrition, irradiation with blue or natural light.

In the preferred embodiment of the invention the fungal biomass in harvested from a submerged fermentation of Aspergillus niger producing Glucoamylase enzyme or citric acid. In another preferred embodiment of the invention the biomass is harvested from a submerged fermentation of Trichoderma longibrachiatum (=T. reesei) or Penicillim funiculosum or Chrysosporium lucknowense (=Myceliophthora thermophile), cultivated to produce cellulase, beta-glucanase or xylanase enzymes. In yet another embodiment of this invention the biomass is harvested from a submerged fermentation of Aspergillus, Trichoderma, Penicillium or Myceliophthora, which are converted into genetically modified hosts to produce and secrete peptide products, such as Cellulase, Phytase or Oxidoreductase enzyme, or secondary metabolites.

After the fermentation the biomass with filter aid is collected by vacuum drum filtration, without antimicrobials (Proxel) added, dried using a fluid bed or belt or spray drier and milled to <50 mkm particles using a hammer mill and optionally micronized to <10 mkm particles using an orbital mill.

In a more preferred embodiment the fungal biomass from any of the fermentations described above is separated from the cultural filtrate on a vacuum drum filter and subjected to a optional heat treatment at 50-70° C. for a period of time ranging from 1 min to 60 min in a wet state. The biomass with filter aid is deposited into a thin 3-inch layer on smooth solid floor, such as concrete, preheated to 30° C. by steaming and is subjected to aeration and exposure to strong artificial light or natural light with periodic turning over the layer by spade or using a mechanical device for 6-12 hours. As an option, a solution of urea is sprayed on top of the layer to provide Nitrogen nutrition. Alternatively, a solution of Glucose is spayed to provide Carbon nutrition. The biomass can be optionally mixed in the beginning of the incubation in the thin layer with a solid substrate, either inert (such as Zeolite) or supporting further fungal growth, such as wheat bran, rice bran, wheat hulls, coffee cake, sunflower cake, sugar beet pulp, wheat midds, whole wheat grain, wheat germ, and the like. The substrate can be optionally supplemented with carbon source, such as glucose, lactose or starch, and a Nitrogen source, such as urea or ammonium sulfate. If the layer overheats because of fast burning of the Carbon source, Carbon source feed is reduced and the layer is cooled down to 30° C. by spraying water and providing a tangential flow of temperate (15-30° C.) air with low (40%-60%) humidity. In addition, the concrete floor can be equipped with a network of underground water pipes, providing cooling by circulating cold water. Incubation is preferably stopped before sporulation starts. Typically after 6-12 hours of incubation under the conditions above the fungal biomass is allowed to dry slightly in place, collected by hand or using a machine, optionally subjected to mechanical, chemical or enzymatic treatment and is sent to fluid bed drying. The dried material is milled to <50 mkm particles using a hammer mill and optionally micronized to <10 nm particles using an orbital mill.

In another preferred embodiment of the invention the biomass is grown in a dedicated fermentation using cost efficient media and the conditions maximizing biomass accumulation and, optionally, conditions maximizing chitin and hydrophobin production. The fermentation could be a conventional solid state fermentation, known in the art. Alternatively, in the most preferred embodiment, the biomass is first maximized during a submerged fermentation stage with maximal aeration on a nutrient-rich medium, optionally supplied with Biotin and Niacin, followed by a short solid state fermentation, with duration sufficient to maximize the biosynthesis of biomass components responsible for mycotoxin binding, such as Chitin, Chitozan and Hydrophobins. In particular, after 2-4 days of submerged fermentation, when a point of diminishing returns in total biomass accumulation is reached, the biomass is harvested using methods known in the art and applied in a wet state to a solid state incubation described above (in the section about processing third-party biomass by-products). Same conditions as far as temperature, cooling, nutrients, illumination and humidity can be maintained. Due to a short period of solid state fermentation and prevalence of the biomass of species of interest, the proposed method has an advantage over conventional solid state fermentation methods of less risk of microbial contamination. This way the SSF stage can be conducted in simplified semi-sterile conditions. After 6-12 hours of incubation under these conditions the fungal biomass is allowed to dry slightly in place, collected by hand or using a machine, optionally subjected to mechanical, chemical or enzymatic treatment and is sent to fluid bed drying. The dried material is milled to <50 mkm particles using a hammer mill and optionally micronized to <10 mkm particles using an orbital mill.

In another embodiment of the invention, the mycotoxin binding capacity of the fungal biomass is enhanced by optional physical treatment (such as micronization and/or porous structure strengthening) and/or chemical treatment (such as generation of additional anion-exchange groups or additional centers of hydrophobicity).

The examples of physical treatment include thermal shock in wet state, illumination and supplementation with structure strengthening agents.

The examples of porous structure strengthening include treatments, when porous and high specific surface structure of the biomass of filamentous fungi is prevented from collapsing on itself and on other components of feed. This alleviates the risk especially pronounced during palletizing and extrusion. Protection of the surface and the pore structure of the fabricated adsorbent is achieved by creating an artificial non-melting layer on the surface and inside the pores using methods known in the art, such as depositing an artificial layer of mineral sediment generated by impregnation of the adsorbent with one soluble compound and then acting upon it with another soluble compound, with both compounds forming upon mixing an insoluble compound in place.

The examples of chemical post-treatment of the biomass of filamentous fungi include autolysis, chemical and enzymatic partial hydrolysis, treatment with organic solvents, acidic and alkaline treatment. From this range, treatment with alkali, protease, chitinase and cellulase are the most preferred.

As the final stage of the treatment, the biomass of filamentous fungi is dried using a belt drier with subsequent dry milling or using a spray drier, with optional subsequent micronization.

Additional objects, advantages and other novel features of the invention will be set forth in part in the description that follows and in part will become apparent to those skilled in the art upon examination of the following or may be learned with the practice of the invention. The objects and advantages of the invention may be realized and obtained by means of the instrumentalities and combinations particularly pointed out in the appended claims.

To achieve the foregoing and other objects, and in accordance with the purposes of the present invention as described herein, a novel method is described for binding mycotoxins present in animal feeds. In particular, in a preferred embodiment, the invention provides a method and a composition for binding mycotoxins present in animal feed rations, human food and agricultural settings encompassing an ingredient based on the modified biomass of filamentous fungi and a conventional non-proprietary mycotoxin adsorbing ingredient, or multiple ingredients, known in the art.

The modified biomass of filamentous fungi can be produced using a number of methods from a number of sources, implying collection of biomass alone or along with other non-soluble residues, that were part of the fermentation, heat treatment in a wet state, partial biomass autolysis or hydrolysis using external enzymes (cellulose, chitinase, protease and similar) or partial hydrolysis using alkali. The non-proprietary mycotoxin binding components, selected from the class of natural clays, artificial clays, organic polymers, activates charcoal, yeast cell wall polysaccharides and such, are readily available from a variety of commercial sources.

In the preferred embodiment of the present invention, the resulting mycotoxin adsorbing component based on biomass of filamentous fungi becomes the core ingredient, enabling the successful expansion of the range of bound mycotoxins, including those difficult to adsorb mycotoxins typical for Northern climates (OTA, T-2, DON, NIV). Other ingredients, providing affinity towards more easily bound mycotoxins typical for Southern climates (AF, FUM, ZEN) can be included at a rate of 10-90% (w/w), chosen from conventional non-proprietary binding agents known in the art and used in the industry, such as, but not limited to: natural clays, man-made clays, organic polymers and yeast cell wall components.

In a preferred embodiment, the composition of the present invention comprises between about 10% and about 90% of the component based on biomass of filamentous fungi, and between about 90% and about 10% of a conventional non-proprietary mycotoxin binding agent. A preferred composition of the invention comprises from between about 25% to about 50% of component based on biomass of filamentous fungi, and between about 75% and about 50% of a conventional non-proprietary mycotoxin binding agent. An especially preferred embodiment of the invention comprises from between about 30% to about 40% of filamentous fungi biomass-based component, and between about 70% and about 60% of a conventional non-proprietary mycotoxin binding agent. The preferred physical form of the invention is a dry, free-flowing powder or micro-granulated powder, suitable for direct inclusion into animal feeds or as a supplement to a total mixed ration.

The compositions provided by the present invention can be added to any commercially available feedstuffs for livestock or companion animals including, but not limited to, premixes, concentrates and pelleted concentrates. The composition provided by the present invention may be incorporated directly into commercially available mashed and pelleted feeds or fed supplementally to commercially available feeds. When incorporated directly into animal feeds, composition of the present invention may be added to such feeds in amounts ranging from 0.2 to about 5 kilograms per ton of feed. In a preferred composition, the invention is added to feeds in amounts ranging from 0.5 to about 2 kilograms per ton of feed. In an especially preferred composition, the invention is added to feeds in amounts ranging from 1 to 2 kilograms per ton of feed. The composition contained in the present invention may be fed to any animal, including but not limited to, avian, bovine, porcine, equine, ovine, caprine, canine, and feline species, aquaculture objects.

The proposed methods of binding of an extended range of mycotoxins are especially useful for alleviating the effect of mycotoxin concentration while fermenting grains during ethanol and beer fermentations. The resulting wet distiller's grain and dried distiller's grain, including DDGS, has on average a 3-fold increase in mycotoxin content compared to initial materials.

Alternatively, the composition contained in the present invention may be directly fed to animals as a supplement in amounts ranging from 2.0 to 20 grams per animal per day. An especially preferred embodiment comprises feeding the composition contained in the present invention to animals in amounts ranging from 5 to 15 grams per animal per day, depending on the animal species, size of the animal and the type of feedstuff.

The composition can be added to any human food, dry, powder, formed, paste, jelly or liquid or used as a functional nutritional supplement in the form of powder, tablets, paste or suspension, with or without other ingredients. When admixed with food or fed as a supplement, the compositions with their surprisingly increased mycotoxin-binding capacity and expanded mycotoxin type range, decrease intestinal absorption of the mycotoxins by the affected human, thereby improving performance and health.

The key components of the biomass of filamentous fungi responsible for binding of mycotoxins (Chitin, Chitozan and Hydrophobins) are difficult to digest in the gastro intestinal tract of animals and humans and are therefore capable to carry out the function of mycotoxin binder until the bound mycotoxins are safely excreted from the organism.

The methods of the invention comprise increasing binding and removal of mycotoxins from animal feedstuffs, including, but not limited to, Aflatoxin, Zearalenone, Vomitoxin, Fumonisins, T2 toxin, and Ochratoxin, thereby increasing safety and nutritional value of the feed and the overall health and performance of the animal. The compositions of the invention are sufficiently effective in increasing binding of OTA, T-2, DON, NIV, compared to binding obtained with current generation of mycotoxin binders, in addition to binding Aflatoxin, Zearalenone, and Fumonisin, where the current mycotoxin binders already excel.

The composition can be added to agricultural soil, seeds and seedlings to provide the future crop plants a resistance to fungal infections.

### EXAMPLES

The following examples are intended to be illustrative of the invention, and are not to be considered restrictive of the scope of the invention as otherwise described herein.

### Example 1

The mycotoxin binding capacity of the adsorbent candidate was measured by adding 1 mkg of mycotoxin as dry weight to 1 gram of adsorbing component (dry weight) suspended on 5 ml of 0.1 M buffer, providing the required pH. The unbound mycotoxin was quantified in the supernatant after removal of the solids by centrifugation (15,000 g for 10 min). A TLC method was used for mycotoxin assay.

The biomass of Trichoderma longibrachiatum (=T.reesei) was produced by submerged fermentation of a cellulose producing strain in shake flasks at 26° C. on standard dextrose media in four repetitions. After 5 days of fermentation the biomass was harvested by centrifugation, rinsed with saline and dried. The dried biomass was milled to particles not exceeding 50 mkm using a hammer mill. The material was used as a candidate for a mycotoxin binder. The effectiveness of T-2 initial binding and residual (after desorption using a buffer rinse) binding is presented in Table 1.

TABLE 1
In-vitro effectiveness of T-2 mycotoxin adsorption
by the biomass of filamentous fungus Trichoderma reesei
produced in four different biomass versions.

| Protein | T-2 mycotoxin | | T-2 mycotoxin | Residual |
|---|---|---|---|---|
| content in | adsorption,% | | desorption, % | adsorption, % |
| the biomass | pH 7.0 | pH 2.0 | pH 8.0 | pH 2.0 |
| 5.2 | 70.7 | 77.0 | 10.1 | 66.9 |
| 8.2 | 77.0 | 77.0 | 4.1 | 72.8 |
| 10.3 | 70.7 | 70.7 | 10.9 | 59.8 |
| 16.8 | 52.0 | 61.4 | 5.9 | 55.4 |

### Example 2

Fulamentous fungus Fusarium sambucinum Fuckel, strain VKM F-842, was grown under aeration in a 10-liter fermentor containing 7 liters of media of the following composition (g/l): sucrose—18; sugar beet molasses—10; ammonium nitrate—3; KH₂PO₄—2.

Fermentation was conducted at start pH of 5.6, 26° C. and agitation of 400 rpm, providing aeration of 1.0 l/l/min. After 48 hours of cultivation the biomass was collected by centrifuging and freeze-dried.

The dried biomass was micronized using an orbital mill to the particle size 3-5 mkm. The fine material produced was tested in an in-vitro HPLC/MS/MS mycotoxin binding assay established. Conditions included adsorption of three mycotoxins typical for North American and European markets—DON (=vomitoxin), ochratoxin (OTA) and zearalenone (ZEN)—from an aqueous solution, pH 6.5 (0.1 M Na-phosphate buffer), at 37° C. within an hour by 0.5% suspension of the adsorbent candidate. Concentration of each mycotoxin in the mix has been chosen at 1 mg/l (in sum—3.0 mg/l).

Mycotoxin content in the model aqueous solution was measured using HPLC/MS/MS on a C-8 column eluted by a gradient of formiate buffer->acetonitrile. Under these HPLC conditions mycotoxins are eluted from the column in the following sequence: DON—OTA—ZEN. The effectiveness of initial binding of three toxins is presented in Table 2.

TABLE 2
In-vitro effectiveness of adsorption of 3 mycotoxins by the dry-
micronized biomass of filamentous fungus Fusarium sambucinum.

| | % of mycotoxin adsorbed from a | | |
|---|---|---|---|
| Adsorbent candidate, 5 g/l, pH | mixture of 3 toxins, 1 mg/l each | | |
| 6.5, 37° C., 1 hour | DON | OTA | ZEA |
| Mycofix Plus (Biomin, Austria) | 4.8 | 0.1 | 42.9 |
| Mycosorb (Alltech, Ireland) | 55.3 | 16.1 | 62.7 |
| Yeast cell wall | 46.7 | 7.0 | 57.6 |
| Fusarium biomass, micronized to | 54.8 | 7.7 | 80.2 |
| 5 mkm | | | |

What is claimed is:

1. A composition and method useful for: adsorbing and thereby rendering harmless in food, animal feed and during invasion of plants by parasitic fungi a wide range of mycotoxins, including difficult to bind mycotoxins of the Northern climates, comprising 10-100% of biomass of one or more filamentous fungi, optionally milled or micronized in a dry state, where the biomass is produced either as by-product of fungal fermentation or in dedicated processes.

2. Method of supplementation of food, when the effective amount of a composition based on micronized biomass of filamentous fungi is added to any human food, dry, powder, formed, paste, jelly or liquid or used as a functional nutritional supplement in the form of powder, tablets, paste or suspension, with or without other ingredients, rendering harmless a wide spectrum of mycotoxins.

3. Method of supplementation of animal feed intended for agricultural or companion animals belonging to the group of invertebrate and vertebrate aquatic, avian and mammalian (such as bovine, porcine, equine, ovine, caprine, canine, feline) species, when the effective amount of a composition based on micronized biomass of filamentous fungi comprises from about 0.02% to about 0.5% by weight of the animal's daily feed ration, rendering harmless a wide spectrum of mycotoxins, including the ones currently difficult to bind.
